(19) United States
(12) Patent Application Publication
(10) Pub. No.: US 2024/0310355 A1
(43) Pub. Date: Sep. 19, 2024

(54) METHOD FOR DETERMINING AN ANALYTE OF INTEREST BY FREQUENCY DETECTION

(71) Applicant: Roche Diagnostics Operations, Inc., Indianapolis, IN (US)

(72) Inventors: Martin Rempt, Penzberg (DE); Christian Wellner, Iffeldorf (DE)

(21) Appl. No.: 18/672,786

(22) Filed: May 23, 2024

Related U.S. Application Data
(63) Continuation of application No. PCT/EP2022/082822, filed on Nov. 22, 2022.

(30) Foreign Application Priority Data
Nov. 24, 2021 (EP) 21210216.4

(51) Int. Cl. G01N 33/487

(52) U.S. Cl. CPC G01N 33/48721

(57) ABSTRACT

The present invention relates to a method for determining an analyte of interest by frequency detection and the use thereof, a modified nanopore, an analyzing system, a kit and the uses thereof.

### FIELD OF THE INVENTION

The present invention relates to a method for determining an analyte of interest by frequency detection and the use thereof, a modified nanopore, an analyzing system, a kit and the uses thereof.

### BACKGROUND OF THE INVENTION

Biologically active components, such as small molecules, proteins, antigens, immunoglobulins, and nucleic acids, are involved in numerous biological processes and functions. Hence, any disturbance in the level of such components can lead to disease or accelerate the disease process. For this reason, much effort has been expended in developing reliable methods to rapidly detect and identify biologically active components for use in patient diagnostics and treatment. For example, detecting a protein or small molecule in a blood or urine sample can be used to assess a patient's metabolic state. Similarly, detection of an antigen in a blood or urine sample can be used to identify pathogens to which a patient has been exposed, thus facilitating an appropriate treatment. It is further beneficial to be able to determine the concentration of an analyte in solution. For example, determining the concentration of a blood or urine component can allow the component to be compared to a reference value, thus facilitating further evaluation of a patient's health status.

A numerous detection and identification methods are available. For example, US202110088511 describes a basic concept of a captured tag by biotin-streptavidin interaction and a bulky protein within a nanopore to give a charged construct which is trapped within a single nanopore. The respective tag is designed to allow a distinct ion current through the pore based upon the degree of blockage within the pore. The insertion within the pore is based upon the protein and/or chemical surrounding on top of the pore and its respective behaviour and/or interactions with the surrounding media.

Normal levels for the AC current are in the range of 1 Hz if sequencing experiments are done and 1000 Hz of read out frequencies.

To modify nanopores have also been seen for sequencing techniques (e.g. Genia) in which enzymes (e.g. polymerases) have been tagged to the nanopore itself.

With this system an open channel after pore insertion, a trapping of the construct of biotin-streptavidin-tag-protein and further modification events can be seen by different current levels. The current levels occur through the respective AC. The switching of the AC (Alternating Current, positive/negative) gives two current levels which reflects the status of the pore and the respective ion flow strength and direction (positive charged ions to negative electrode and vice versa).

The current read out concepts for the (AC) values by doing experiments is very valuable if only one analyte needs to be detected. The used buffer compositions can be tuned very carefully so that it is often hard to interpret raw signals of the trapped construct and can be optimized. The current concepts do not incorporate a concept of the detection for the identity of the tag or further binding and/or changing events of the construct, this would be in fact of high interest if multiplexing is desired and the digital counting for active binding/pore sites and bounded sites/pores. Furthermore, nanopores due to their single molecule nature can give fuzzy/hard to interpret signals and therefore it cannot be examined how many working pores there are with desired accuracy. This is important if also low and higher concentrated analytes are present which is only possible if the numbers of free binding sites, the binder as well as covered binding sites can be count and/or determined.

The binding of an analyte or in general a modification event is only seen in the electrical current level shift. This shift is often misleading and needs to be better specified by another dimension of information.

There is, however, still a need of improving the detection and identification methods.

The present invention relates to a method for determining an analyte of interest such as steroids, peptides, proteins, and other types of analytes, e.g. in biological samples by frequency detection. The present invention further relates to a modified nanopore, an analyzing system and uses thereof.

It is an object of the present invention to provide a method for determining an analyte of interest by frequency detection, the use thereof, a modified nanopore, an analyzing system and the use thereof.

This object is or these objects are solved by the subject matter of the independent claims. Further embodiments are subjected to the dependent claims.

### SUMMARY OF THE INVENTION

In the following, the present invention relates to the following aspects:

In a first aspect, the present invention relates to a method for determining an analyte of interest by frequency detection comprising the steps of:
a) Providing a nanopore, wherein the nanopore is embedded in a two dimensional material and has a first resonant frequency f₁, wherein an AC current having an AC frequency f_{AC} is applied,
b) Providing a modified nanopore, wherein the modified nanopore is embedded in the two dimensional material and has a second resonant frequency f₂, wherein the AC current having the AC frequency f_{AC} is applied, wherein f_{AC}>f₁>f₂, wherein the modified nanopore comprises the analyte of interest,
c) Detecting a frequency shift Δ of the first and the second frequency, and
d) Determining the analyte of interest by using the frequency shift Δ.

In a second aspect, the present invention relates to the use of the method of the first aspect of the invention for determining the analyte of interest.

In a third aspect, the present invention relates to a modified nanopore using in a method of the first aspect of the invention for determining the analyte of interest comprising:
a protein nanopore or a solid-state nanopore,
optionally a binder, and
the analyte of interest.

In a fourth aspect, the present invention relates to an analyzing system comprising
a nanopore, wherein the nanopore is embedded in a two dimensional material and has a first resonant frequency f₁, wherein an AC current having an AC frequency f_{AC} is applied,
a modified nanopore, wherein the modified nanopore is embedded in the two dimensional material and has a second resonant frequency f₂, wherein the AC current having the AC frequency f_{AC} is applied, wherein f_{AC}>f₁>f₂,
wherein the modified nanopore comprises the analyte of interest, and wherein the analyzing system is configured to detect a frequency shift Δ of the first and the second frequency and to determine the analyte of interest by using the frequency shift Δ.

In a sixth aspect, the present invention relates a kit suitable to perform a method of the first aspect of the invention comprising
a reagent or reagents for forming the two dimensional material,
a reagent or reagents for forming the nanpore,
a reagent or reagents for forming the modified nanpore, and
optionally components that are selected from the group consisting of calibrator, buffer, additive, consumable, algorithm for evaluation and combinations thereof.

In a seventh aspect, the present invention relates the use of kit of the fifth aspect of the present invention for determining an analyte of interest by frequency detection.

### LIST OF FIGURES

FIGS. 1A, 2 and 4 show the general principle of an experimented current/magnetic driven readout of a modified nanopore (Biotin-Streptavidin-Protein within a lipid bilayer) and time dependent readout shown.

FIG. 1B shows the used tag to generate a defined voltage level in the nanopore.

FIG. 3 shows the method according to the workflow from a nanopore which is influenced by an external field (magnetic or electric) and therefore deflected which results in a changing current.

FIG. 5A describes the RAW signals (current vs. time) of a modified nanopore. FIG. 5B describes the respective signals (amplitude vs. frequency) of the in FIG. 5A described areas after using Fourier Transformation of the raw signals mentioned from FIG. 5A (FFT signal).

FIG. 6 describes the RAW signals (current vs. time) and the FFT signals (amplitude vs. frequency) of a similar experiment like FIG. 5.

FIGS. 7A, 7B, 8A to 8E, 9A to 9C and 10A to 10B describe the RAW signals (current vs. time) and the FFT signals (amplitude vs. frequency) of a modified nanopore. FIG. 9A to 9C describes the frequency dependent FFT analysis @ f_{AC}=1 Hz. FIG. 10A to 10B describes the frequency dependent FFT analysis @ f_{AC}=1000 Hz.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps. The term “including” and “comprising” can be used interchangeably.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10, . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In the context of the present disclosure, the term “analyte”, “analyte molecule”, or “analyte(s) of interest” are used interchangeably referring the chemical species to be analysed. Chemical species, i.e. analytes, can be any kind of molecule present in a living organism, include but are not limited to nucleic acid (e.g. DNA, mRNA, miRNA, rRNA etc.), amino acids, peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system.

The term “determining” the the analyte of interest, as used herein refers to the quantification and/or qualification of the analyte of interest, e.g. to determining the presence of the analyte of interest and/or measuring the level of the analyte of interest in the sample.

The term “nanopore” can mean a tiny pore in the nanometer scale sitting on a thin membrane or two dimensional material, e.g. a lipid bilayer or is formed out as tiny pore in the nanometer scale from a solid 2D plane (e.g. Glass-Nanopore). Nanopore an pore can be used interchangeably.

The term “construct” as used herein is a broad term and is to be given its ordinary and customary meaning to a person of ordinary skill in the art and is not to be limited to a special or customized meaning. The term specifically may refer, without limitation, to the modified nanopore embedded in the two dimensional material, wherein the modification can be induced by a binder, which is attached, e.g. covalently or non-covalently, to the nanopore to form the modified nanopore.

The term “amphiphilic molecules” as used herein is a broad term and is to be given its ordinary and customary meaning to a person of ordinary skill in the art and is not to be limited to a special or customized meaning. The term specifically may refer, without limitation, to any compound that contains two distinct covalently bonded components with different affinity for the solvent in the same molecule, in which one part possesses a high affinity for polar solvents (such as water), and another part has a strong affinity for nonpolar solvents, such as hydrocarbons, ethers, and esters. Surfactants, polymer amphiphiles, and some lipid molecules, containing both hydrophilic and hydrophobic components, are typical examples of amphiphilic molecules.

The term “bilayer” as used herein is a broad term and is to be given its ordinary and customary meaning to a person of ordinary skill in the art and is not to be limited to a special or customized meaning. The term specifically may refer, without limitation, to a membrane made of two layers of lipid molecules, preferably phospholipid molecules.

A “clinical diagnostics system” is a laboratory automated apparatus dedicated to the analysis of samples for in vitro diagnostics. The clinical diagnostics system may have different configurations according to the need and/or according to the desired laboratory workflow. Additional configurations may be obtained by coupling a plurality of apparatuses and/or modules together. A “module” is a work cell, typically smaller in size than the entire clinical diagnostics system, which has a dedicated function. This function can be analytical but can be also pre-analytical or post analytical or it can be an auxiliary function to any of the pre-analytical function, analytical function or post-analytical function. In particular, a module can be configured to cooperate with one or more other modules for carrying out dedicated tasks of a sample processing workflow, e.g. by performing one or more pre-analytical and/or analytical and/or post-analytical steps. In particular, the clinical diagnostics system can comprise one or more analytical apparatuses, designed to execute respective workflows that are optimized for certain types of analysis, e.g. clinical chemistry, immunochemistry, coagulation, hematology, liquid chromatography separation, mass spectrometry, etc. Thus the clinical diagnostic system may comprise one analytical apparatus or a combination of any of such analytical apparatuses with respective workflows, where pre-analytical and/or post analytical modules may be coupled to individual analytical apparatuses or be shared by a plurality of analytical apparatuses. In alternative pre-analytical and/or post-analytical functions may be performed by units integrated in an analytical apparatus. The clinical diagnostics system can comprise functional units such as liquid handling units for pipetting and/or pumping and/or mixing of samples and/or reagents and/or system fluids, and also functional units for sorting, storing, transporting, identifying, separating, detecting.

The clinical diagnostic system can comprise a nanopore holder. The nanopore holder is known for a skilled person and thus not explained in detail, e.g. Bhatti et al., RSC Adv., 2021, 11, 28996.

The clinical diagnostic system can further comprise a detector for detecting the frequency shift Δ of the first and the second frequency.

The clinical diagnostic system can further comprise a transformer or calculator for calculating the first frequency, second frequency and/or frequency shift from step e) from a time domain into a frequency domain. The transformer is configured to use a transferring operation, e.g. Fourier Transformation.

A “sample preparation station” can be a pre-analytical module coupled to one or more analytical apparatuses or a unit in an analytical apparatus designed to execute a series of sample processing steps aimed at removing or at least reducing interfering matrix components in a sample and/or enriching analytes of interest in a sample. Such processing steps may include any one or more of the following processing operations carried out on a sample or a plurality of samples, sequentially, in parallel or in a staggered manner: pipetting (aspirating and/or dispensing) fluids, pumping fluids, mixing with reagents, incubating at a certain temperature, heating or cooling, centrifuging, separating, filtering, sieving, drying, washing, resuspending, aliquoting, transferring, storing, etc.).

A “kit” is any manufacture (e.g., a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

In this detailed description, references to “one embodiment”, “an embodiment”, or “in embodiments” mean that the feature being referred to is included in at least one embodiment of the technology with regards to all its aspects according to present disclosure. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the technology in all its aspects according to present disclosure can include any variety of combinations and/or integrations of the embodiments described herein.

### Embodiments

In a first aspect, the present invention relates to a method for determining an analyte of interest by frequency detection comprising the steps of:
a) Providing a nanopore, wherein the nanopore is embedded in a two dimensional material and has a first resonant frequency f₁, wherein an AC current having an AC frequency f_{AC} is applied,
b) Providing a modified nanopore, wherein the modified nanopore is embedded in the two dimensional material and has a second resonant frequency f₂, wherein the AC current having the AC frequency f_{AC} is applied,
wherein f_{AC}>f₁>f₂,
wherein the modified nanopore comprises the analyte of interest,
c) Detecting a frequency shift Δ of the first and the second frequency, and
d) Determining the analyte of interest by using the frequency shift Δ.

It is described herein the principles to detect frequency changes for a charged construct trapped within a nanopore within a two dimensional material, e.g. a lipid bilayer. It is a detection of a tagged nanopore construct (modified nanopore) visible and a very stable signal after switching from the time domain to the frequency domain is shown.

Preferably, the “eigenfrequency” or resonant frequency of the two dimensional material-nanopore-construct (f₁ and/or f₂) should not matched the driving AC frequency f_{AC} due to the fact that destruction by resonance disaster events which means that lipid bilayers are formed well but no pore construct is established.

Driving force can be current driven or possibly also magnetic driven.

The nanopore itself can also been possibly used without a trapped construct by covalently binding a protein or a nonspecific binder onto the nanopore itself.

According to step (a), the nanopore is provided. The nanopore is embedded in a two dimensional material. The nanopore has a first resonant frequency f₁. An AC current having an AC frequency f_{AC} is applied.

The abbreviation f₁ and f1 can be used interchangeably for the first resonant frequency.

In embodiments of the first aspect of the invention, the method for determining an analyte of interest by frequency detection comprising the steps of:
a) Providing a nanopore, wherein the nanopore is embedded in a two dimensional material and has a first resonant frequency f₁, wherein an AC current having an AC frequency f_{AC} is applied,
b) Providing a modified nanopore, wherein the modified nanopore is embedded in the two dimensional material and has a second resonant frequency f₂,
wherein the AC current having the AC frequency f_{AC} is applied,
wherein f_{AC}>f₁>f₂,
wherein the modified nanopore comprises the analyte of interest,
c) Detecting a frequency shift Δ of the first and the second frequency, wherein Δ=|f₂|−|f₁|, and
d) Determining the analyte of interest by using the frequency shift Δ.

In embodiments of the first aspect of the invention, the first resonant frequency f₁ is the resonant frequency of the nanopore.

In embodiments of the first aspect of the invention, the first resonant frequency f₁ is the resonant frequency of the nanopore embedded in the two dimensional material.

Preferably, more than one nanopore is provided, e.g. 2, 3, 4, or several hundreds or thousands or million nanopores are provided. The nanopores are provided on a well plate. The well plat is a semiconductor chip. The semiconductor chip can have 128 k cells.

In principle, nanopores are known for a skilled person in the art and thus are not explained into much detail. Nanopores can be purchased by Genia Technologies. To measure current through a nanopore, a complementary metal oxide semiconductor (CMOS) chip containing e.g. 264 individually addressable electrodes can be used. This chip was developed by Genia Technologies.

In embodiments of the first aspect of the invention, the nanopore comprises a pore having a pore size of less than 1000 nm, preferably having a pore size between 2 nm and 20 nm.

In embodiments of the first aspect of the invention, the nanopore is a protein nanopore or a solid-state nanopore.

In embodiments of the first aspect of the invention, the protein nanopore is α-haemolysin or Mycobacterium smegmatis porin A.

In embodiments of the first aspect of the invention, the protein nanopore is a synthetic membrane comprising MoS₂, graphene, Si, SiNx and/or SiO₂.

According to step (b), the modified nanopore is provided. The modified nanopore is embedded in the two dimensional material. The modified nanopore has a second resonant frequency f₂. The AC current having the AC frequency f_{AC} is applied, wherein f_{AC}>f₁>f₂. The modified nanopore comprises the analyte of interest. In particular, the same AC current having the AC frequency f_{AC} is applied to the nanopore as well as to the modified nanopore.

In embodiments of the first aspect of the invention, the AC frequency f_{AC} measurement is paralyzed in time and in space and/or is multiplexed in time and in space.

In embodiments of the first aspect of the invention, the AC frequency f_{AC} measurement is performed by using electrical and/or magnetic measurement.

The abbreviation f_{AC} and f_{AC} can be used interchangeably for the AC frequency. The abbreviation f₂ and f2 can be used interchangeably for the second resonant frequency. The resonant frequency (f1 and/or f2) can also be named as eigenfrequency.

In embodiments of the first aspect of the invention, the second resonant frequency f₂ is the resonant frequency of the modified nanopore.

In embodiments of the first aspect of the invention, the second resonant frequency f₂ is the resonant frequency of the modified nanopore embedded in the two dimensional material.

The modified nanopore or the modified nanopore embedded in the two dimensional material can be named here and in the whole disclosure as construct.

Preferably, more than one modified nanopores are provided, e.g. 2, 3, 4, or several hundreds or thousands modified nanopores are provided. The modified nanopores are provided on a well plate.

In embodiments of the first aspect of the invention, the modified nanopore and the nanopore differentiate from each other by the modification. The modification can be a complex or construct, which is trapped within the nanopore to form the modified nanopore. Additionally or as an alternative, the modification can be a binder, which is attached to the nanopore to form the modified nanopore.

In embodiments of the first aspect of the invention, the modified nanopore provided in step b) is produced by modifying the nanopore, which is provided in step a).

In embodiments of the first aspect of the invention, the modified nanopore is produced by an analyte comprising a construct, which is trapped within the nanopore.

In embodiments of the first aspect of the invention, the construct comprises an aptamere, an antibody, an antibody fragment, e.g. FAB, an unspecific binder, e.g. Van der Waals interaction between C18 and/or aromatic structures with the analyte, a DNA, a enzyme e.g. polymerase, an ionic interaction, e.g. positive charged and negatively charged pairs.

In embodiments of the first aspect of the invention, the modified nanopore is produced by a binder attached to the nanopore.

In embodiments of the first aspect of the invention, the binder is covalently attached to the nanopore.

In embodiments of the first aspect of the invention, the binder is attached to the nanopore nonspecifically by non-covalent bonds, preferably Van der Waals forces.

In embodiments of the first aspect of the invention, the modified nanopore comprises a pore having a pore size of less than 1000 nm, preferably having a pore size between 2 nm and 20 nm.

In embodiments of the first aspect of the invention, the modified nanopore is a protein nanopore or a solid-state nanopore.

In embodiments of the first aspect of the invention, the protein modified nanopore is α-haemolysin or Mycobacterium smegmatis porin A.

In embodiments of the first aspect of the invention, the protein modified nanopore is a synthetic membrane comprising MoS₂, graphene, Si, SiNx and/or SiO₂.

In embodiments of the first aspect of the invention, the AC frequency is more than the first resonant frequency and more than the second resonant frequency. The first resonant frequency is more than the second resonant frequency and smaller than the AC frequency. The second resonant frequency is smaller the first resonant frequency and smaller than the first resonant frequency.

In embodiments of the first aspect of the invention, f_{AC}>f₁>f₂ is |f_{AC}|>|f₁|>|f₂|. In principle, the absolute value can be left out, because the general meaning is that every increase in mass (e.g. nanopore or modified nanopore) leads directly to a reduction in the resonance frequency, as the whole part becomes heavier.

In embodiments of the first aspect of the invention, the analyte of interest is a single-molecule of the modified nanopore. Preferably, exact one single molecule of the analyte is part of the modified nanopore. Thus, this method allows a single molecule detection.

In embodiments of the first aspect of the invention, the frequency shift is: Δ=f₂−f₁, preferably Δ=|f₂|−|f₁|. In particular, both terms can be uses interchangeably.

In embodiments of the first aspect of the invention, the AC frequency f_{AC} is more than 500 Hz or 650 Hz or 650 Hz or 700 Hz or 750 Hz, e.g. 770 Hz.

In embodiments of the first aspect of the invention, the AC frequency f_{AC} is less than 2000 Hz or 1700 Hz or 1500 Hz or 1300 Hz or 1000 Hz, e.g. 900 Hz.

In embodiments of the first aspect of the invention, the first and/or second frequency is between 400 Hz and 500 Hz. In principle, other first and/or second frequency are possible depending on the kind of analyte of interest, the nanopore and/or the two dimensional material.

In embodiments of the first aspect of the invention, the two dimensional material comprises amphiphilic molecules. In embodiments of the first aspect of the invention, the two dimensional material is a bilayer, preferably a lipid bilayer.

In embodiments of the first aspect of the invention, the two dimensional material is a monolayer, preferably comprising an amphiphilic molecule having two hydrophilic groups or two hydrophobic groups, more preferably wherein the two hydrophilic groups are separated from each other by a hydrophobic group or wherein the two hydrophobic groups are separated from each other by a hydrophilic group.

In embodiments of the first aspect of the invention, the analyte of interest is a small molecule.

In embodiments of the first aspect of the invention, the analyte of interest is a protein.

In embodiments of the first aspect of the invention, the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

In embodiments of the first aspect of the invention, the method comprises further steps before step c):
e) Detecting the voltage as a function of time of the nanopore provided in step a) and the modified nanopore provided in step b), and/or
f) Calculating the first frequency, second frequency and/or frequency shift from step e) from a time domain into a frequency domain by using a transferring operation, e.g. Fourier Transformation.

In embodiments of the first aspect of the invention, the steps c) and d) are performed after steps a) and b). Preferably, step a) is performed before step b). More preferably, the nanopore in step a) is provided and then this nanopore provided in step a) is modified to form the modified nanopore provided in step b).

In embodiments of the first aspect of the invention, the analyte comprises biological tissue, biological material, eatable goods, polymers, paintings, archaeological artifacts, artificial bone, skin, urine, or blood.

In embodiments of the first aspect of the invention, the analyte is a protein, e.g. TNT.

In embodiments of the first aspect of the present invention, the analyte molecule comprising one or more keto groups is a ketosteroid. In particular embodiments of the first aspect of the present invention, the ketosteroid is selected from the group consisting of testosterone, epitestosterone, dihydrotestosterone (DHT), desoxymethyltestosterone (DMT), tetrahydrogestrinone (THG), aldosterone, estrone, 4-hydroxyestrone, 2-methoxyestrone, 2-hydroxyestrone, 16-ketoestradiol, 16-alpha-hydroxyestrone, 2-hydroxyestrone-3-methylether, prednisone, prednisolone, pregnenolone, progesterone, dehydroepiandrosterone (DHEA), 17-hydroxypregnenolone, 17-hydroxyprogesterone, androsterone, epiandrosterone, Δ4-androstenedione, 11-deoxycortisol, corticosterone, 21-deoxycortisol, 11-deoxycorticosterone, allopregnanolone and aldosterone.

In embodiments of the first aspect of the present invention, the analyte molecule comprising one or more carboxyl groups is selected from the group consisting of Δ8-tetrahydrocannabinolic acid, benzoylecgonin, salicylic acid, 2-hydroxybenzoic acid, gabapentin, pregabalin, valproic acid, vancomycin, methotrexate, mycophenolic acid, montelukast, repaglinide, furosemide, telmisartan, gemfibrozil, diclofenac, ibuprofen, indomethacin, zomepirac, isoxepac and penicillin. In embodiments of the first aspect of the present invention, the analyte molecule comprising one or more carboxyl groups is an amino acid selected from the group consisting of arginine, lysine, aspartic acid, glutamic acid, glutamine, asparagine, histidine, serine, threonine, tyrosine, cysteine, tryptophan, alanine, isoleucine, leucine, methionine, phenyalanine, valine, proline and glycine.

In embodiments of the first aspect of the present invention, the analyte molecule comprising one or more aldehyde groups is selected from the group consisting of pyridoxal, N-acetyl-D-glucosamine, alcaftadine, streptomycin and josamycin.

In embodiments of the first aspect of the present invention, the carbonyl group is an carbonyl ester group. In embodiments of the first aspect of the present invention, the analyte molecule comprising one or more ester groups is selected from the group consisting of cocaine, heroin, Ritalin, aceclofenac, acetylcholine, amcinonide, amiloxate, amylocaine, anileridine, aranidipine artesunate and pethidine.

In embodiments of the first aspect of the present invention, the analyte molecule comprising one or more anhydride groups is selected from the group consisting of cantharidin, succinic anhydride, trimellitic anhydride and maleic anhydride.

In embodiments of the first aspect of the present invention, the analyte molecule comprising one or more diene groups is a secosteroid. In embodiments, the secosteroid is selected from the group consisting of cholecalciferol (vitamin D3), ergocalciferol (vitamin D2), calcifediol, calcitriol, tachysterol, lumisterol and tacalcitol. In particular, the secosteroid is vitamin D, in particular vitamin D2 or D3 or derivates thereof. In particular embodiments, the secosteroid is selected from the group consisting of vitamin D2, vitamin D3, 25-hydroxyvitamin D2, 25-hydroxyvitamin D3 (calcifediol), 3-epi-25-hydroxyvitamin D2, 3-epi-25-hydroxyvitamin D3, 1,25-dihydroxyvitamin D2, 1,25-dihydroxyvitamin D3 (calcitriol), 24,25-dihydroxyvitamin D2, 24,25-dihydroxyvitamin D3. In embodiments of the first aspect of the present invention, the analyte molecule comprising one or more diene groups is selected from the group consisting of vitamin A, tretinoin, isotretinoin, alitretinoin, natamycin, sirolimus, amphotericin B, nystatin, everolimus, temsirolimus and fidaxomicin.

In embodiments of the first aspect of the present invention, the analyte molecule comprises a single hydroxyl group or two hydroxyl groups. In embodiments, more than one hydroxyl group is present in the analyte molecule, the two hydroxyl groups may be positioned adjacent to each other (1,2-diol) or may be separated by 1, 2 or 3 C atoms (1,3-diol, 1,4-diol, 1,5-diol, respectively). In particular embodiments of the first aspect, the analyte molecule comprises a 1,2-diol group. In embodiments, wherein only one hydroxyl group is present, said analyte is selected from the group consisting of primary alcohol, secondary alcohol and tertiary alcohol. In embodiments of the first aspect of the present invention, wherein the analyte molecule comprises one or more hydroxyl groups, the analyte is selected from the group consisting of benzyl alcohol, menthol, L-carnitine, pyridoxine, metronidazole, isosorbide mononitrate, guaifenesin, clavulanic acid, Miglitol, zalcitabine, isoprenaline, aciclovir, methocarbamol, tramadol, venlafaxine, atropine, clofedanol, alpha-hydroxyalprazolam, alpha-Hydroxytriazolam, lorazepam, oxazepam, Temazepam, ethyl glucuronide, ethylmorphine, morphine, morphine-3-glucuronide, buprenorphine, codeine, dihydrocodeine, p-hydroxypropoxyphene, O-desmethyltramadol, Desmetramadol, dihydroquinidine and quinidine. In embodiments of the first aspect of the present invention, wherein the analyte molecule comprises more than one hydroxyl groups, the analyte is selected from the group consisting of vitamin C, glucosamine, mannitol, tetrahydrobiopterin, cytarabine, azacitidine, ribavirin, floxuridine, Gemcitabine, Streptozotocin, adenosine, Vidarabine, cladribine, estriol, trifluridine, clofarabine, nadolol, zanamivir, lactulose, adenosine monophosphate, idoxuridine, regadenoson, lincomycin, clindamycin, Canagliflozin, tobramycin, netilmicin, kanamycin, ticagrelor, epirubicin, doxorubicin, arbekacin, streptomycin, ouabain, amikacin, neomycin, framycetin, paromomycin, erythromycin, clarithromycin, azithromycin, vindesine, digitoxin, digoxin, metrizamide, acetyldigitoxin, deslanoside, Fludarabine, clofarabine, gemcitabine, cytarabine, capecitabine, vidarabine, and plicamycin.

In embodiments of the first aspect of the present invention, the analyte molecule comprises one or more thiol group (including but not limited to alkyl thiol and aryl thiol groups) as a functional group. In embodiments of the first aspect of the present invention, the analyte molecule comprising one or more thiol groups is selected from the group consisting of thiomandelic acid, DL-captopril, DL-thiorphan, N-acetylcysteine, D-penicillamine, glutathione, L-cysteine, zofenoprilat, tiopronin, dimercaprol, succimer.

In embodiments of the first aspect of the present invention, the analyte molecule comprises one or more disulfide group as a functional group. In embodiments of the first aspect of the present invention, the analyte molecule comprising one or more disulfide groups is selected from the group consisting of glutathione disulfide, dipyrithione, selenium sulfide, disulfiram, lipoic acid, L-cystine, fursultiamine, octreotide, desmopressin, vapreotide, terlipressin, linaclotide and peginesatide. Selenium sulfide can be selenium disulfide, SeS₂, or selenium hexasulfide, Se₂S₆.

In embodiments of the first aspect of the present invention, the analyte molecule comprises one or more epoxide group as a functional group. In embodiments of the first aspect of the present invention, the analyte molecule comprising one or more epoxide groups is selected from the group consisting of Carbamazepine-10,11-epoxide, carfilzomib, furosemide epoxide, fosfomycin, sevelamer hydrochloride, cerulenin, scopolamine, tiotropium, tiotropium bromide, methylscopolamine bromide, eplerenone, mupirocin, natamycin, and troleandomycin.

In embodiments of the first aspect of the present invention, the analyte molecule comprises one or more phenol groups as a functional group. In particular embodiments of the first aspect of the present invention, analyte molecules comprising one or more phenol groups are steroids or steroid-like compounds. In embodiments of the first aspect of the present invention, the analyte molecule comprising one or more phenol groups is a steroid or a steroid-like compound having an A-ring which is sp² hybridized and an OH group at the 3-position of the A-ring. In particular embodiments of the first aspect of the present invention, the steroid or steroid-like analyte molecule is selected from the group consisting of estrogen, estrogen-like compounds, estrone (E1), estradiol (E2), 17a-estradiol, 17b-estradiol, estriol (E3), 16-epiestriol, 17-epiestriol, and 16, 17-epiestriol and/or metabolites thereof. In embodiments, the metabolites are selected from the group consisting of estriol, 16-epiestriol (16-epiE3), 17-epiestriol (17-epiE3), 16,17-epiestriol (16,17-epiE3), 16-ketoestradiol (16-ketoE2), 16a-hydroxyestrone (16a-OHEI), 2-methoxyestrone (2-MeOE1), 4-methoxyestrone (4-MeOE1), 2-hydroxyestrone-3-methyl ether (3-MeOE1), 2-methoxyestradiol (2-MeOE2), 4-methoxyestradiol (4-MeOE2), 2-hydroxyestrone (2-OHE1), 4-hydroxyestrone (4-OHE1), 2-hydroxyestradiol (2-OHE2), estrone (E1), estrone sulfate (E1s), 17a-estradiol (E2a), 17b-estradiol (E2B), estradiol sulfate (E2S), equilin (EQ), 17a-dihydrocquilin (EQa), 17b-dihydroequilin (EQb), Equilenin 17-dihydroequilenin (ENa), 17α-dihydroequilenin, 17β-dihydroequilenin (ENb), Δ8,9-dehydroestrone (dE1), Δ8,9-dehydroestrone sulfate (dE1s), Δ9-tetrahydrocannabinol, mycophenolic acid. β or b can be used interchangeably. α and a can be used interchangeably.

In embodiments of the first aspect of the present invention, the analyte molecule comprises an amine group as a functional group. In embodiments of the first aspect of the present invention, the amine group is an alkyl amine or an aryl amine group. In embodiments of the first aspect of the present invention, the analyte comprising one or more amine groups is selected from the group consisting of proteins and peptides. In embodiments of the first aspect of the present invention, the analyte molecule comprising an amine group is selected from the group consisting of 3,4-methylenedioxyamphetamine, 3,4-methylenedioxy-N-ethylamphetamine, 3,4-methylenedioxymethamphetamine, Amphetamine, Methamphetamine, N-methyl-1,3-benzodioxolylbutanamine, 7-aminoclonazepam, 7-aminoflunitrazepam, 3,4-dimethylmethcathinone, 3-fluoromethcathinone, 4-methoxymethcathinone, 4-methylethcathinone, 4-methylmethcathinone, amfepramone, butylone, ethcathinone, clephedrone, methcathinone, methylone, methylenedioxypyrovalerone, benzoylecgonine, dehydronorketamine, ketamine, norketamine, methadone, normethadone, 6-acetylmorphine, diacetylmorphine, morphine, norhydrocodone, oxycodone, oxymorphone, phencyclidine, norpropoxyphene, amitriptyline, clomipramine, dothiepin, doxepin, imipramine, nortriptyline, trimipramine, fentanyl, glycylxylidide, lidocaine, monocthylglycylxylidide, N-acetylprocainamide, procainamide, pregabalin, 2-Methylamino-1-(3,4-methylendioxyphenyl)butan, N-methyl-1,3-benzodioxolylbutanamine, 2-Amino-1-(3,4-methylendioxyphenyl)butan, 1,3-benzodioxolylbutanamine, normeperidine, O-Destramadol, desmetramadol, tramadol, lamotrigine, Theophylline, amikacin, gentamicin, tobramycin, vancomycin, Methotrexate, Gabapentin sisomicin and 5-methylcytosine.

In embodiments of the first aspect of the present invention, the analyte molecule is a carbohydrate or substance having a carbohydrate moiety, e.g. a glycoprotein or a nucleoside. In embodiments of the first aspect of the present invention, the analyte molecule is a monosaccharide, in particular selected from the group consisting of ribose, desoxyribose, arabinose, ribulose, glucose, mannose, galactose, fucose, fructose, N-acetylglucosamine, N-acetylgalactosamine, neuraminic acid, N-acetylneurominic acid, etc. In embodiments, the analyte molecule is an oligosaccharide, in particular selected from the group consisting of a disaccharide, trisaccharid, tetrasaccharide, polysaccharide. In embodiments of the first aspect of the present invention, the disaccharide is selected from the group consisting of sucrose, maltose and lactose. In embodiments of the first aspect of the present invention, the analyte molecule is a substance comprising above described mono-, di-, tri-, tetra-, oligo- or polysaccharide moiety.

In embodiments of the first aspect of the present invention, the analyte molecule comprises an azide group as a functional group which is selected from the group consisting of alkyl or aryl azide. In embodiments of the first aspect of the present invention, the analyte molecule comprising one or more azide groups is selected from the group consisting of zidovudine and azidocillin.

Such analyte molecules may be present in biological or clinical samples such as body liquids, e.g. blood, serum, plasma, urine, saliva, spinal fluid, etc., tissue or cell extracts, etc. In embodiments of the first aspect of the present invention, the analyte molecule(s) are present in a biological or clinical sample selected from the group consisting of blood, serum, plasma, urine, saliva, spinal fluid, and a dried blood spot. In some embodiments of the first aspect of the present invention, the analyte molecules may be present in a sample which is a purified or partially purified sample, e.g. a purified or partially purified protein mixture or extract.

Surprisingly, it was found that with the present invention the frequency changes for a charged construct trapped within a nanopore within a lipid bilayer can be detected. By modulation the AC frequencies, “eigenfrequency” of the lipid bilayer/nanopore construct are even higher frequencies. It is shown that a detection of a tagged nanopore construct is visible and a very stable signal after switching from the time domain to the frequency domain can be observed.

In embodiments of the first aspect of the present invention, the modified nanopore comprises:
a protein nanopore or a solid-state nanopore,
optionally a binder, and
the analyte of interest.

In a second aspect, the present invention relates to the use of the method of the first aspect of the present invention for determining the analyte of interest.

All embodiments mentioned for the first aspect of the invention apply for the second aspect of the invention and vice versa.

In a third aspect, the present invention relates to a modified nanopore using in a method of the first aspect of the present invention for determining the analyte of interest comprising:
a protein nanopore or a solid-state nanopore,
optionally a binder, and
the analyte of interest.

All embodiments mentioned for the first aspect of the invention and/or second aspect of the invention apply for the third aspect of the invention and vice versa.

In a fourth aspect, the present invention relates to an analyzing system comprising
a nanopore, wherein the nanopore is embedded in a two dimensional material and has a first resonant frequency f₁, wherein an AC current having an AC frequency f_{AC} is applied,
a modified nanopore, wherein the modified nanopore is embedded in the two dimensional material and has a second resonant frequency f₂, wherein the AC current having the AC frequency f_{AC} is applied,
wherein f_{AC}>f₁>f₂,
wherein the modified nanopore comprises the analyte of interest, and
wherein the analyzing system is configured to detect a frequency shift Δ of the first and the second frequency and to determine the analyte of interest by using the frequency shift Δ.

All embodiments mentioned for the first aspect of the invention and/or second aspect of the invention and/or third aspect of the invention apply for the fourth aspect of the invention and vice versa.

In embodiments of the fourth aspect of the present invention, the analyzing system is a clinical diagnostics system.

In a fifth aspect, the present invention relates to the use of the analyzing system for determining an analyte of interest by frequency detection.

In embodiments of the fourth aspect of the present invention, the AC current is adapted or fixed to the experimental conditions, e.g. buffer conditions, type of pore, type of analyte.

All embodiments mentioned for the first aspect of the invention and/or second aspect of the invention and/or third aspect of the invention and/or fourth aspect of the invention apply for the fifth aspect of the invention and vice versa.

In a sixth aspect, the present invention relates a kit suitable to perform a method of the first aspect of the invention comprising
a reagent or reagents for forming the two dimensional material,
a reagent or reagents for forming the nanpore,
a reagent or reagents for forming the modified nanpore, and
optionally components that are selected from the group consisting of calibrator, buffer, additive, consumable, algorithm for evaluation and combinations thereof.

All embodiments mentioned for the first aspect of the invention and/or second aspect of the invention and/or third aspect of the invention, fourth aspect of the invention and/or fifth aspect of the invention apply for the sixth aspect of the invention and vice versa.

In a seventh aspect, the present invention relates the use of kit of the fifth aspect of the present invention for determining an analyte of interest by frequency detection.

All embodiments mentioned for the first aspect of the invention and/or second aspect of the invention and/or third aspect of the invention, fourth aspect of the invention, fifth aspect of the invention and/or sixth aspect of the invention apply for the seventh aspect of the invention and vice versa. In further embodiments, the present invention relates to the following aspects:

1. A method for determining an analyte of interest by frequency detection comprising the steps of:
a) Providing a nanopore, wherein the nanopore is embedded in a two dimensional material and has a first resonant frequency f₁, wherein an AC current having an AC frequency f_{AC} is applied,
b) Providing a modified nanopore, wherein the modified nanopore is embedded in the two dimensional material and has a second resonant frequency f₂,
wherein the AC current having the AC frequency f_{AC} is applied,
wherein f_{AC}>f₁>f₂,
wherein the modified nanopore comprises the analyte of interest,
c) Detecting a frequency shift Δ of the first and the second frequency, and
d) Determining the analyte of interest by using the frequency shift Δ.

2. The method of aspect 1 comprising further steps before step c):
e) Detecting the voltage as a function of time of the nanopore provided in step a) and the modified nanopore provided in step b), and
f) Calculating the first frequency, second frequency and/or frequency shift from step e) from a time domain into a frequency domain by using a transferring operation, e.g. Fourier Transformation.

3. The method of any of the proceeding aspects, wherein the modified nanopore provided in step b) is produced by modifying the nanopore, which is provided in step a).

4. The method of any of the proceeding aspects, wherein the analyte of interest is a single-molecule of the modified nanopore.

5. The method of any of the proceeding aspects, wherein the frequency shift is: Δ=f₂−f₁, and/or wherein the AC frequency f_{AC} measurement is paralyzed in time and in space.

6. The method of any of the proceeding aspects, wherein the AC frequency f_{AC} is more than 500 Hz or 650 Hz or 650 Hz or 700 Hz or 750 Hz, e.g. 770 Hz.

7. The method of any of the proceeding aspects, wherein the AC frequency f_{AC} is less than 2000 Hz or 1700 Hz or 1500 Hz or 1300 Hz or 1000 Hz, e.g. 900 Hz.

8. The method of any of the proceeding aspects, wherein the first and/or second frequency is between 400 Hz and 500 Hz.

9. The method of any of the proceeding aspects, wherein the modified nanopore is produced by an analyte comprising a construct, which is trapped within the nanopore.

10. The method of any of the proceeding aspects, wherein the construct comprises an aptamere, an antibody, an antibody fragment, e.g. FAB, an unspecific binder, e.g. Van der Waals interaction between C18 and/or aromatic structures with the analyte, a DNA, a enzyme e.g. polymerase.

11. The method of any of the proceeding aspects, wherein the modified nanopore is produced by a binder attached to the nanopore.

12. The method of any of the proceeding aspects, wherein the binder is covalently attached to the nanopore.

13. The method of any of the proceeding aspects, wherein the binder is attached to the nanopore nonspecifically by non-covalent bonds, preferably Van der Waals forces.

14. The method of any of the proceeding aspects, wherein the nanopore comprises a pore having a pore size of less than 1000 nm, preferably having a pore size between 2 nm and 20 nm.

15. The method of any of the proceeding aspects, wherein the nanopore is a protein nanopore or a solid-state nanopore.

16. The method of any of the proceeding aspects, wherein the protein nanopore is α-haemolysin or Mycobacterium smegmatis porin A.

17. The method of any of the proceeding aspects, wherein the protein nanopore is a synthetic membrane comprising MoS₂, Graphene, Si, SiNx and/or SiO₂.

18. The method of any of the proceeding aspects, wherein the two dimensional material comprises amphiphilic molecules.

19. The method of any of the proceeding aspects, wherein the two dimensional material is a bilayer, preferably a lipid bilayer.

20. The method of any of the proceeding aspects, wherein the two dimensional material is a monolayer, preferably comprising an amphiphilic molecule having two hydrophilic groups or two hydrophobic groups, more preferably wherein the two hydrophilic groups are separated from each other by a hydrophobic group or wherein the two hydrophobic groups are separated from each other by a hydrophilic group.

21. The method of any of the proceeding aspects, wherein the analyte of interest is a small molecule.

22. The method of any of the proceeding aspects, wherein the analyte of interest is a protein.

23. The method of any of the proceeding aspects, wherein the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

24. The method of any of the proceeding aspects, wherein the modified nanopore comprises:
a protein nanopore or a solid-state nanopore,
optionally a binder, and
the analyte of interest.

25. Use of the method of any of the proceeding aspects for determining the analyte of interest.

26. A modified nanopore using in a method of any of the proceeding aspects for determining the analyte of interest comprising:
a protein nanopore or a solid-state nanopore,
optionally a binder, and
the analyte of interest.

27. An analyzing system comprising
a nanopore, wherein the nanopore is embedded in a two dimensional material and has a first resonant frequency f₁, wherein an AC current having an AC frequency f_{AC} is applied,
a modified nanopore, wherein the modified nanopore is embedded in the two dimensional material and has a second resonant frequency f₂, wherein the AC current having the AC frequency f_{AC} is applied,
wherein f_{AC}>f₁>f₂,
wherein the modified nanopore comprises the analyte of interest, and
wherein the analyzing system is configured to detect a frequency shift Δ of the first and the second frequency and to determine the analyte of interest by using the frequency shift Δ.

28. Use of the analyzing system of aspect 27 for determining an analyte of interest by frequency detection.

29. A kit suitable to perform a method of any of the proceeding aspects comprising
a reagent or reagents for forming the two dimensional material,
a reagent or reagents for forming the nanpore,
a reagent or reagents for forming the modified nanpore (e.g. tags), and
optionally components that are selected from the group consisting of calibrator, buffer, additive, consumable (e.g. chips, pipettes), algorithm for evaluation and combinations thereof.

30. Use of the kit of aspect 29 for determining an analyte of interest by frequency detection.

### Examples

The following examples are provided to illustrate, but not to limit the presently claimed invention.

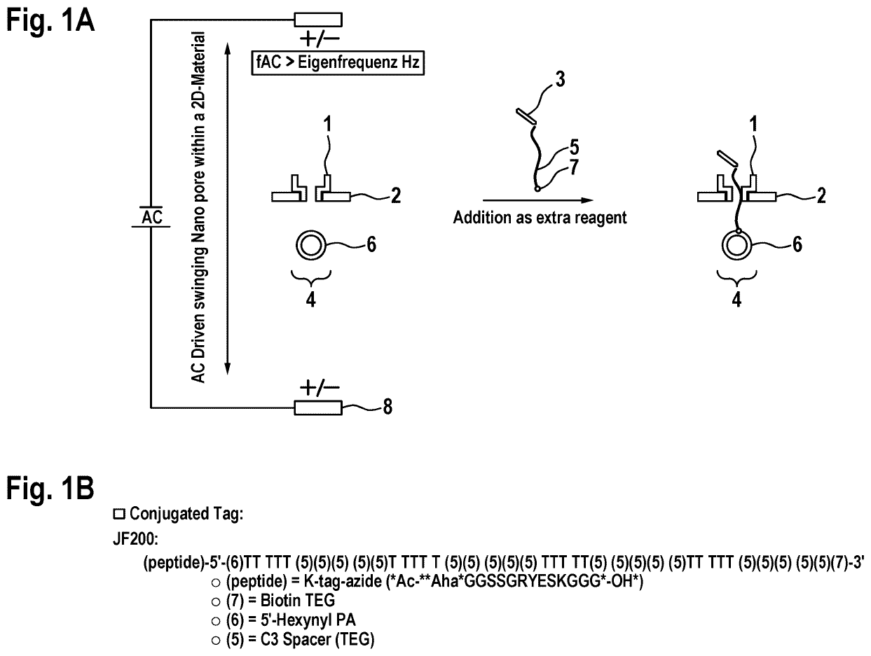

FIG. 1A shows the method according to the first aspect of the invention. It is shown a method according to one embodiment. It is the general principle of an experimented current driven readout of a modified nanopore (Biotin-Streptavidin-Protein within a lipid bilayer) and time dependent readout shown.

A nanopore is provided 1, wherein the nanopore 1 is embedded in a two dimensional material 2, e.g. a lipid bilayer, and has a first resonant frequency f₁. An AC current having an AC frequency f_{AC}. A modified nanopore 4 is provided which is in this case the nanopore 1 adapted by modification. The modified nanopore 4 has a second resonant frequency f₂. A low AC pulse with frequency to around eigenfrequency of the modified nanopore or modified nanopore-two dimensional material is applied. FID (free induction decay) of the vibration is based on the inertia.

As an example, an open nanopore 1 is electrically inserted in the lipid bilayer 2. On the trans side of the nanopore the buffer contains streptavidin. After a certain time period an analyte of interest, e.g. a fab-fragment 3 which is covalently attached to a modified oligonucleotide 5 equipped with a biotin 7 label is applied at the cis side of the nanopore.

The Fab-Oligo Streptavidin molecule is entering the nanopore, gets attached to the streptavidin and therefore cannot escape the nanopore anymore. A AC frequency is applied the whole time over the experiment.

The modified nonpore 4 comprises the nanopore 1 and the analyte of interest 3. The analyte of interest 3 can be e.g. a protein or small molecule, wherein a biotin 5 can be tagged. It is: f_{AC}>f₁>f₂.

Then, the frequency shift Δ of the first and the second frequency is detected and the analyte of interest by using the frequency shift Δ is determined. In particular, the voltage as a function of time of the nanopore provided in step a) and the modified nanopore provided in step b) are detected and the first frequency, second frequency and/or frequency shift from step e) is calculated from a time domain into a frequency domain by using a transferring operation, e.g. Fourier Transformation.

The modified nanopore can be prepared as follows:

FIG. 1B shows the used tag to generate a defined voltage level in the nanopore. It comprises or consists of C3 Spacer and Thymidines. In principle, other tags, e.g. DNA, RNA, Peptide etc. are possible. The nanopore can be alpha Hämolysin or a mutation thereof. In principle, other nanopores are possible which is suitable to be the “host” of the construct. Examples of pore-forming proteins are alpha hemolysin, aerolysin, and MspA porin.

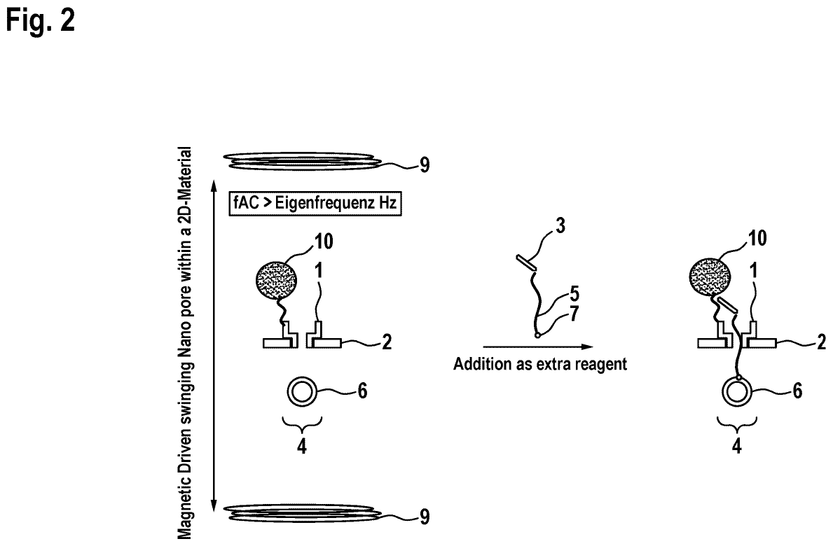

FIG. 2 shows the method according to the first aspect of the invention. Different from FIG. 1A, FIG. 2 describes the procedure that a magnetic label (e.g. magnetic nanoparticle) 10 is covalently attached to the nanopore 1 which is electrically inserted in a two dimensional material, e.g. lipid bilayer 2. Supported from a alternating magnetic field applied by the Helmholtz-Coils 9 the magnetic label can apply the external switching magnetic field to the nanopore within the bilayer.

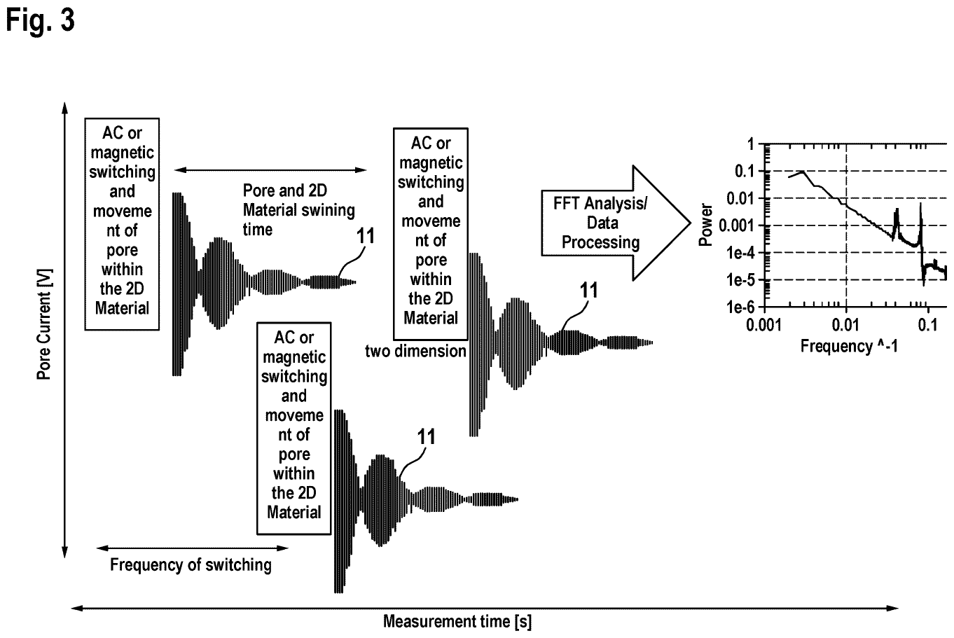

FIG. 3 shows the method according to the workflow from a nanopore which is influenced by an external field (magnetic or electric) and therefore deflected which results in a changing current. A free induction decay 11 after each external field switching event is acquired. The time domain in which the experiment is run (preferably seconds) is changed to the frequency domain is performed using data processing methods e.g. FFT.

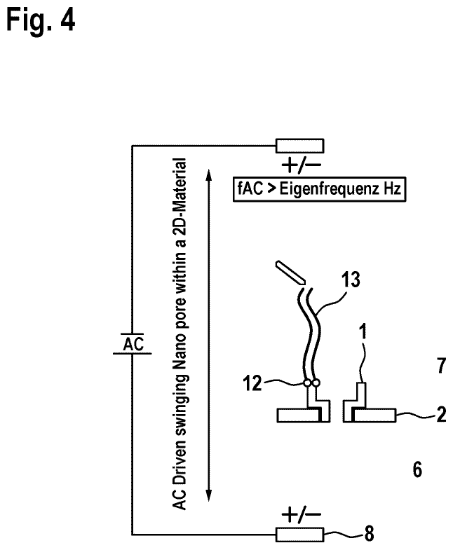

FIG. 4 shows the method according to the first aspect of the invention. It is the general principle of a possible current driven readout of a modified nanopore (Biotin-Streptavidin-Protein within a lipid bilayer) and time dependent readout shown. An open nanopore 1 is electrically inserted in the lipid bilayer 2. The FAB-binding moiety is covalently bound to the nanopore e.g. by L-LNA support 13. An AC frequency is applied the whole time over the experiment. The term “fab” and “FAB” can be used interchangeably. The term “pore” and “nanopore” can be used interchangeably.

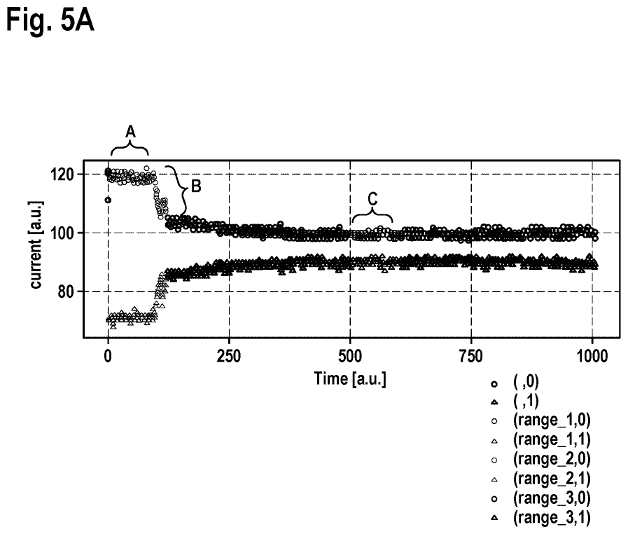

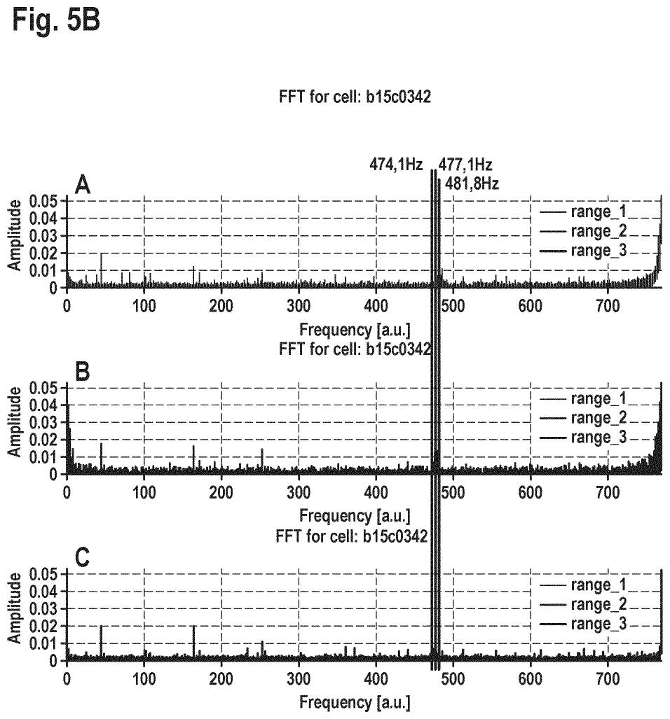

FIG. 5A describes the RAW signals (voltage vs. time) of a modified nanopore (Biotin-Streptavidin-Protein within a lipid bilayer) at an AC frequency for 770 Hz. The x axis is expressed as a time signal (0 corresponds to the start of the experiment and 1000 expressed the end of the experiment). The area A is the open nanopore within the lipid bilayer, area B is the addition of the biotin-oligonucleotide-FAB fragment molecule addition and area C is the steady state of the in area B added chemical bound to a streptavidin. FIG. 5B describes the respective signals (amplitude vs. frequency) of the in FIG. 5A described areas after using Fourier Transformation of the raw signals mentioned from FIG. 5A (FFT signal). A clear peak at around 475 Hz can be seen which is shifting from area A (481.8 Hz) over area B (477.1 Hz) to the steady state area C (474.1 Hz). A differentiation of area A and area C can be seen.

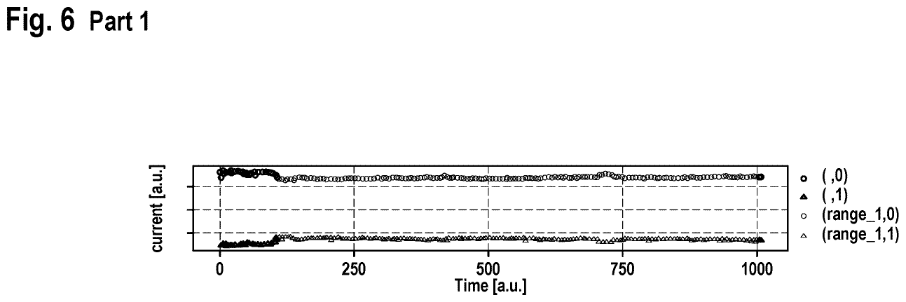

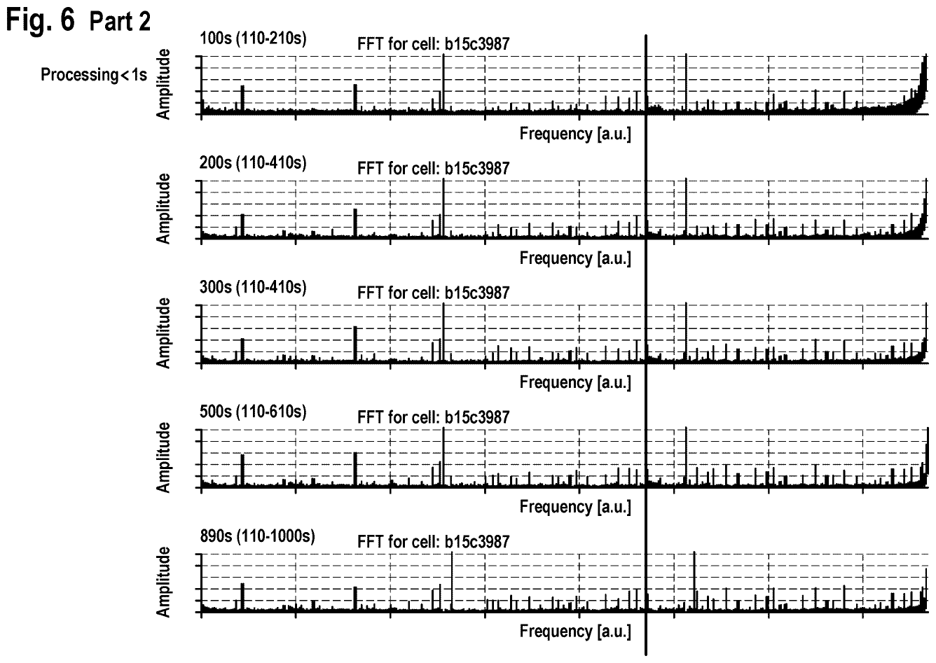

FIG. 6 describes the RAW signals (voltage vs. time) and the FFT signals (amplitude vs. frequency) of a similar experiment like FIG. 5. The FFT analyses have performed with increasing timepoints used for FFT analysis (from 100s to 890s). The FFT signal is not changing therefore the spectra quality can be enhanced using multiple points for FFT or multiplication of resulting FFT spectra.

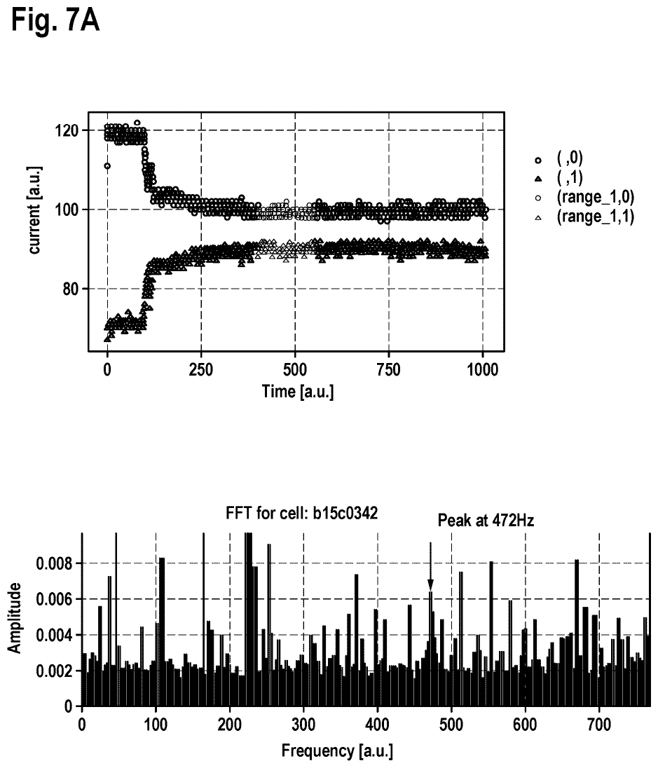

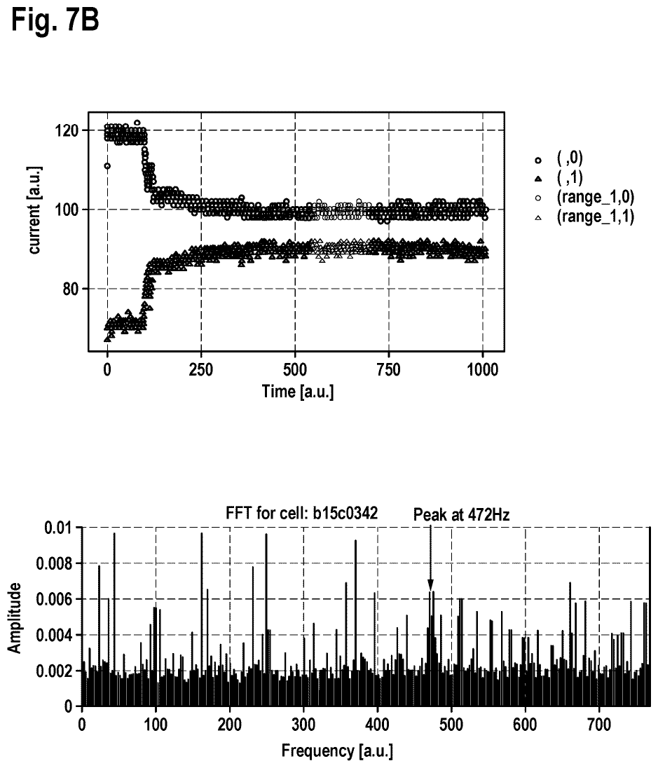

FIG. 7A describes the RAW signals (voltage vs. time) and the FFT signals (amplitude vs. frequency) of a modified nanopore (Biotin-Streptavidin-Protein) within a lipid bilayer at an AC frequency for 770 Hz. The FFT spectra from a certain raw data points is expressed with a peak at 472 Hz.

FIG. 7B describes the RAW signals (voltage vs. time) and the FFT signals (amplitude vs. frequency) of a modified nanopore (Biotin-Streptavidin-Protein) within a lipid bilayer at an AC frequency for 770 Hz. The FFT spectra from a different time point than in FIG. 7A within the same experiment is expressed with a peak at 472 Hz which is exactly the same as for FIG. 7A. Therefore a signal constancy can be assumed in the steady state of the nanopore.

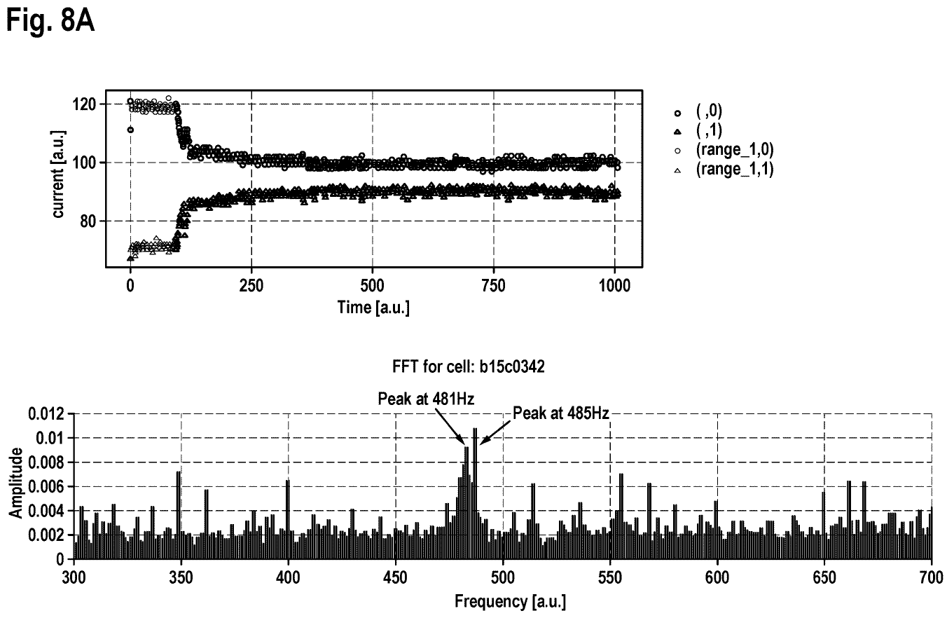

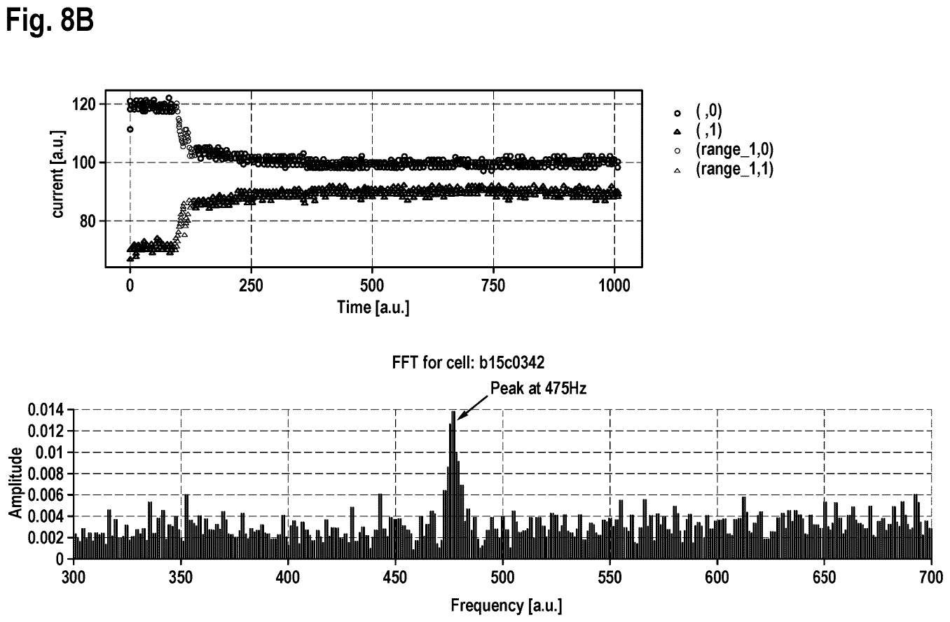

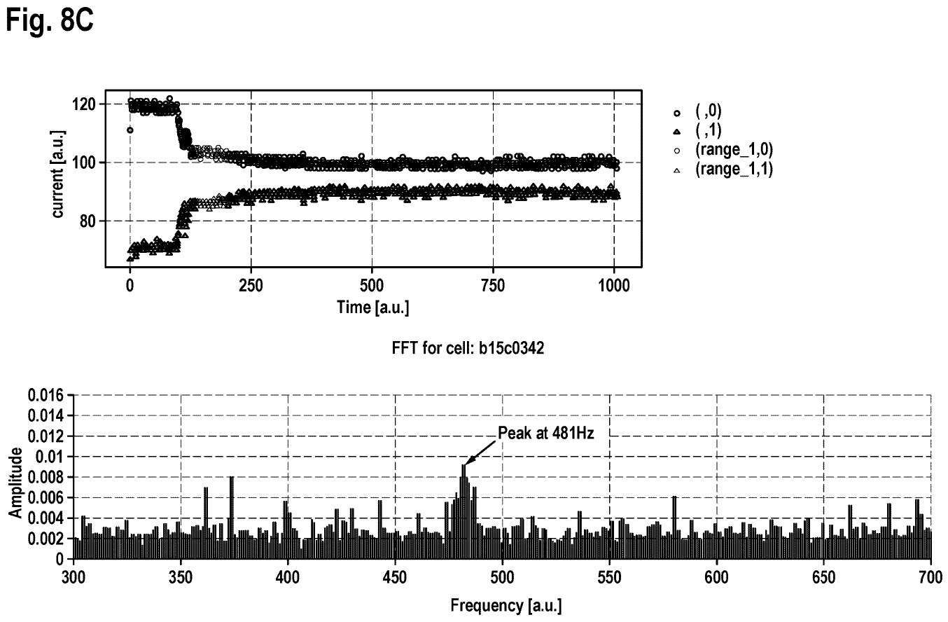

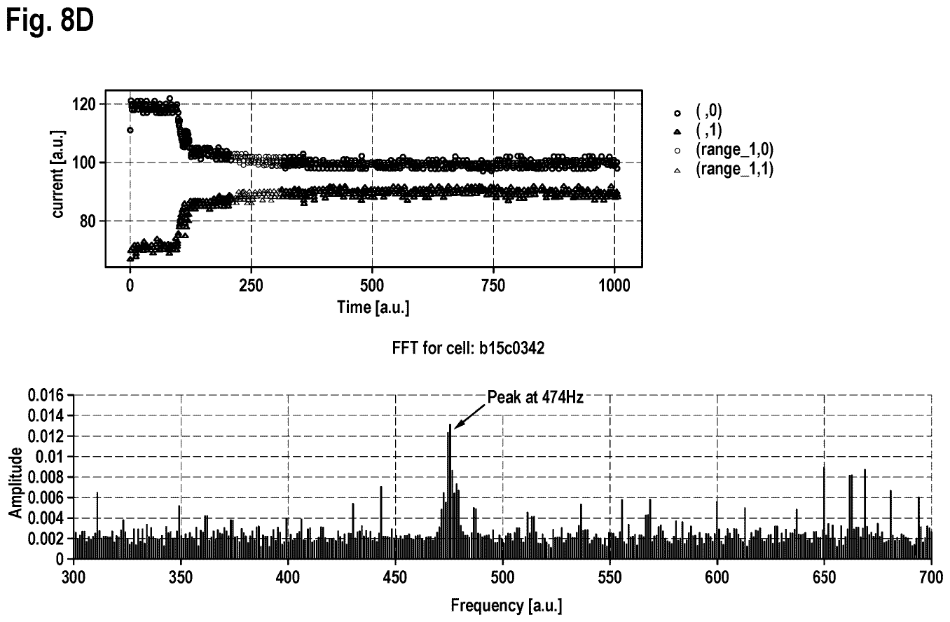

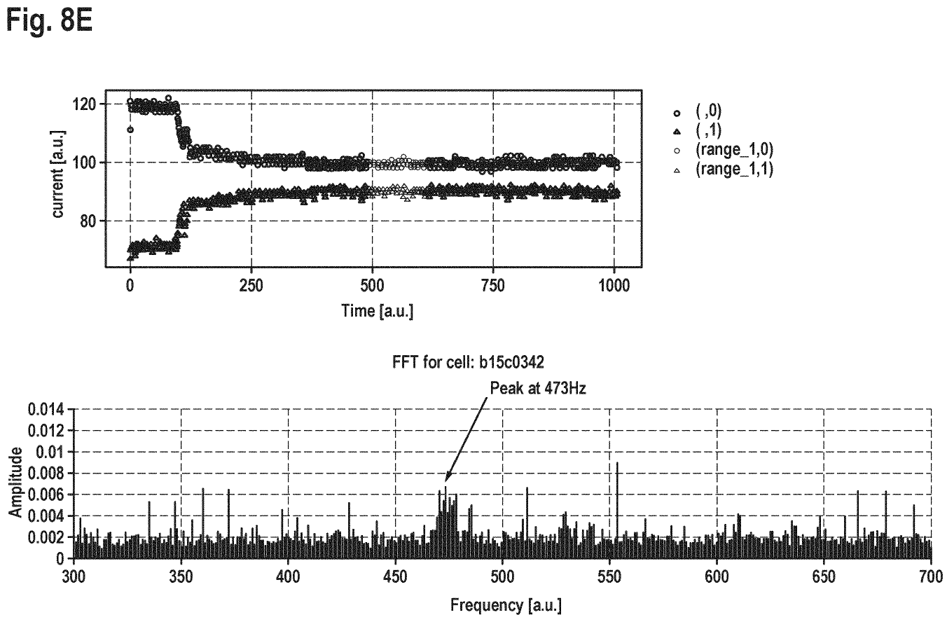

FIGS. 8A to 8E describe the RAW signals (voltage vs. time) and the FFT signals (amplitude vs. frequency) of a modified nanopore (Biotin-Streptavidin-Protein) within a lipid bilayer at an AC frequency for 770 Hz.

At FIG. 8A the area for an open nanopore is processed with FFT analysis to result in a signal for the open pore at 481 Hz.

At FIG. 8B the area for an open/blocked nanopore intermediate in which the biotin tag is not trapped by the streptavidin within the pore is processed with FFT analysis to result in a signal for the open nanopore at 475 Hz.

At FIG. 8C the area for an open nanopore in which the biotin tag FAB molecule has left the pore without binding is processed with FFT analysis to result in a signal for the open pore at 481 Hz.

At FIGS. 8D and 8E the area for a steady stated blocked pore with streptavidin/biotin binding are processed with FFT analysis to result in a signal for blocked nanopore at 474 Hz and 473 Hz, respectively.

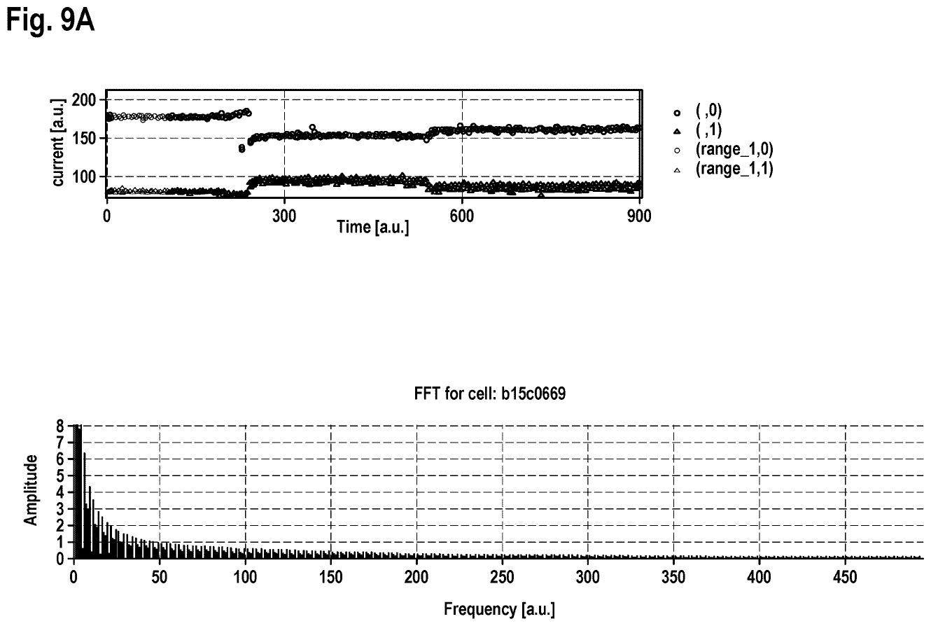

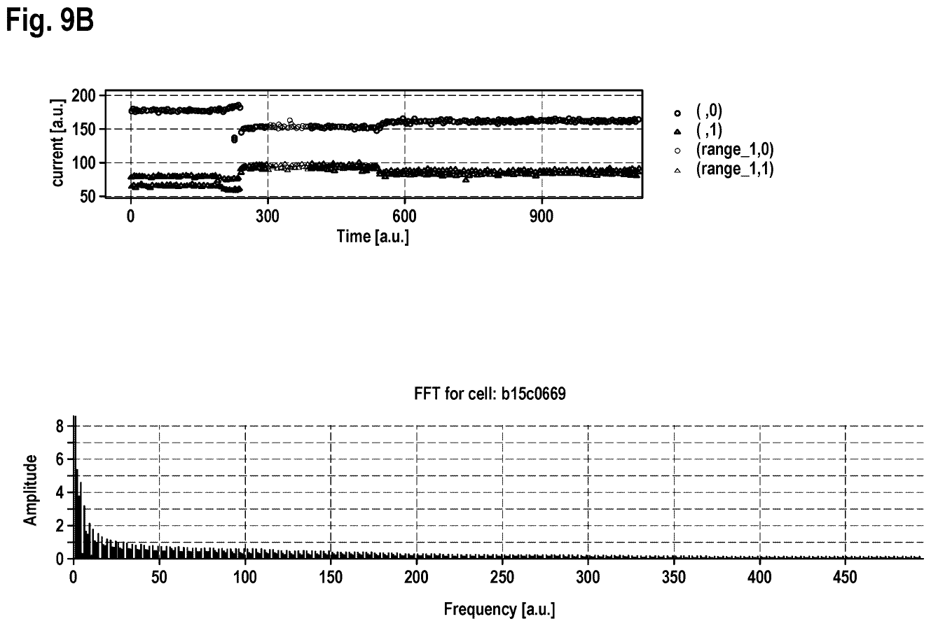

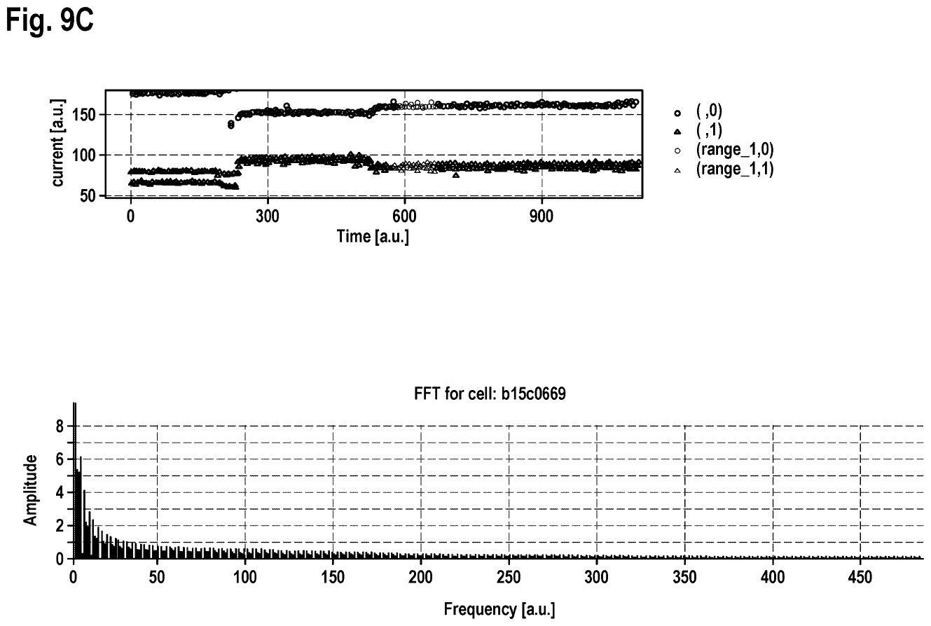

FIGS. 9A to 9C describe the RAW signals (voltage vs. time) and the FFT signals (amplitude vs. frequency) of a modified nanopore (Biotin-Streptavidin-Protein) within a lipid bilayer with a AC frequency at 1 Hz. The FFT spectra from FIG. 9A to 9C are shown at different time points and do not differentiate the states of the experiments as well as do not show any respective eigenfrequency of the nanopore within the lipid bilayer. Therefore a driving frequency>eigenfrequency of the nanopore within the lipid bilayer is necessary.

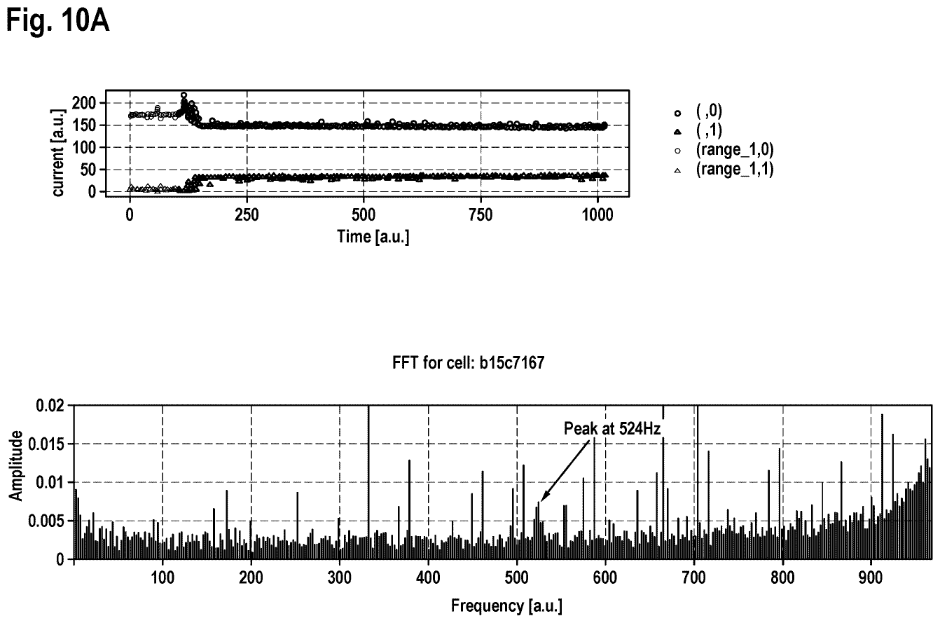

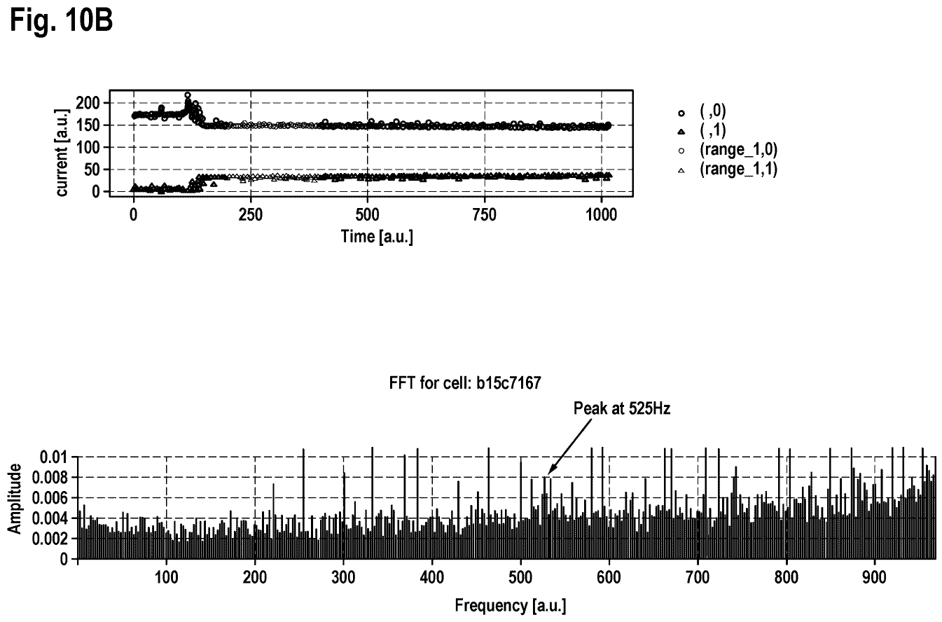

FIGS. 10A to 10B describe the RAW signals (voltage vs. time) and the FFT signals (amplitude vs. frequency) of a modified nanopore (Biotin-Streptavidin-Protein) within a lipid bilayer driven by an AC frequency with 1000 Hz. The FFT spectra from FIG. 10A to 10B at different time points (before and after FAB-Oligo-Biotin molecule flow overt the pore) can not differentiate the states of the experiments (before and after applying the FAB-Oligo-Biotin molecule flow). This is expressed by the fact that before the respective FAB-Oligo-Biotin molecule flow and afterwards the same frequency at around 524 Hz has been observed. The result is in this case that the FAB-Oligo-Biotin molecule has not been cached up within the nanopore but nevertheless the nanopore within the lipid bilayer expresses its respective eigenfrequency at around 524 Hz.

This patent application claims the priority of the European patent application 21210216.4, wherein the content of this European patent application is hereby incorporated by references.

### LIST OF REFERENCES

1—nanopore
2—two dimensional material
3—analyte of interest or analyte binder (e.g. FAB Fragment)
4—modified nanopore
5—polymer (e.g. Oligonucleotide)
6—streptavidin
7—anchoring tag, e.g. Biotin
8—electrical contact
9—Helmholtz Coils
10—magnetic tag (e.g. magnetic nanoparticle)
11—free induction decay
12—covalently binding between polymer(s) (e.g. DNA and/or Protein)
13—rigid polymer (e.g. DNA)

1. A method for determining an analyte of interest by frequency detection, the method comprising:
a) providing a nanopore, wherein the nanopore is embedded in a two dimensional material and has a first resonant frequency f₁, wherein an AC current having an AC frequency f_{AC} is applied;
b) providing a modified nanopore, wherein the modified nanopore is embedded in the two dimensional material and has a second resonant frequency f₂, wherein the AC current having the AC frequency f_{AC} is applied, wherein f_{AC}>f₁>f₂, wherein the modified nanopore comprises the analyte of interest;
c) detecting a frequency shift Δ of the first frequency and the second frequency, wherein Δ=|f₂|−|f₁|; and
d) determining the analyte of interest by using the frequency shift Δ.

2. The method of claim 1, further comprising:
e) detecting, before step c), a voltage as a function of time of the nanopore and the modified nanopore; and
f) converting, before step c), the first frequency, the second frequency and/or the frequency shift from a time domain into a frequency domain by using a transferring operation.

3. The method of claim 1, wherein the modified nanopore is produced by modifying the nanopore.

4. The method of claim 1, wherein the analyte of interest is a single-molecule of the modified nanopore.

5. The method of claim 1, wherein the AC frequency f_{AC} measurement is paralyzed in time and in space.

6. The method of claim 1, wherein the AC frequency f_{AC} is more than 500 Hz.

7. The method of claim 1, wherein the first frequency is between 400 Hz and 500 Hz.

8. The method of claim 1, wherein the modified nanopore is produced by an analyte comprising a construct, which is trapped within the nanopore.

9. The method of claim 1, wherein the modified nanopore is produced by a binder attached to the nanopore.

10. The method of claim 1, wherein the binder is covalently attached to the nanopore.

11. The method of claim 1, wherein the binder is attached to the nanopore by non-covalent bonds.

12. (canceled)

13. The method of claim 1, wherein the modified nanopore comprises a protein nanopore.

14. An analyzing system, comprising:
a nanopore, wherein the nanopore is embedded in a two dimensional material and has a first resonant frequency f₁, wherein an AC current having an AC frequency f_{AC} is applied and
a modified nanopore, wherein the modified nanopore is embedded in the two dimensional material and has a second resonant frequency f₂, wherein the AC current having the AC frequency f_{AC} is applied, wherein f_{AC}>f₁>f₂, wherein the modified nanopore comprises the analyte of interest,
wherein the analyzing system is configured to detect a frequency shift Δ of the first frequency and the second frequency and to determine the analyte of interest by using the frequency shift Δ.

15. (canceled)

16. A kit suitable to perform the method of claim 1, the kit comprising:
one or more reagents for forming the two dimensional material;
one or more reagents for forming the nanpore; and
one or more reagents for forming the modified nanpore.

17. (canceled)

18. The method of claim 1, wherein the AC frequency f_{AC} is less than 2000 Hz.

19. The method of claim 1, wherein the second frequency is between 400 Hz and 500 Hz.

20. The method of claim 1, wherein the modified nanopore comprises a solid state nanopore.

21. The method of claim 2, wherein the transferring operation comprises a Fourier Transformation.

22. The method of claim 11, wherein the binder is attached to the nanospore by Van der Waals forces.
